(19) United States
(12) Patent Application Publication
(10) Pub. No.: US 2022/0308074 A1
(43) Pub. Date: Sep. 29, 2022

(54) QUANTIFICATION METHOD OF VITAMIN D DERIVATIVE, ENZYME FOR QUANTIFICATION, COMPOSITION FOR QUANTIFICATION, KIT FOR QUANTIFICATION, ELECTRODE, SENSOR CHIP, AND SENSOR

(71) Applicant: KIKKOMAN CORPORATION, Noda-shi (JP)

(72) Inventors: Kanako HAYASHI, Noda-shi (JP); Atsushi ICHIYANAGI, Noda-shi (JP)

(21) Appl. No.: 17/837,142

(22) Filed: Jun. 10, 2022

Related U.S. Application Data
(63) Continuation of application No. PCT/JP2020/045938, filed on Dec. 9, 2020.

(30) Foreign Application Priority Data
Dec. 11, 2019 (JP) 2019-224090

(51) Int. Cl. G01N 33/82; G01N 33/52; C12N 9/02

(52) U.S. Cl. CPC G01N 33/82; G01N 33/52; C12N 9/0004

(57) ABSTRACT

A quantitation method of vitamin D derivative is provided including adding an oxidoreductase to a sample. The quantitation method of vitamin D derivative may further include reducing a mediator by adding the oxidoreductase, and reacting the reduced mediator with a reagent to determine a concentration of the vitamin D derivative.

### CROSS-REFERENCE TO RELATED APPLICATIONS

This application is a Continuation of International Patent Application No. PCT/JP2020/045938, filed on Dec. 9, 2020, which claims the benefit of priority to Japanese Patent Application No. 2019-224090, filed on Dec. 11, 2019, the entire contents of which are incorporated herein by reference.

### FIELD

The present invention relates to a quantification method of vitamin D derivative, an enzyme for quantification, a composition for quantification, a kit for quantification, an electrode, a sensor chip, and a sensor.

### BACKGROUND

Vitamin D is a physiologically active substance that acts on a variety of biological processes in the body. Vitamin D includes vitamin D₂ (Ergocalciferol) and vitamin D₃ (Cholecalciferol). The sources of vitamin D are the biosynthesis of vitamin D₃ in the skin and the ingestion of vitamin D₂ and vitamin D₃ from foods, supplements and the like. Since vitamin D₂ and vitamin D₃ are subjected to the same metabolism and have the same function, they are referred to as vitamin D when vitamin D₂ and vitamin D₃ are not distinguished.

Vitamin D taken into the body is hydroxylated in the liver and converted to 25-hydroxyvitamin D, which is stored in hepatocytes. 25-Hydroxyvitamin D is released into the blood in the form of being bound to vitamin D-binding protein. Since the half-life of 25-hydroxyvitamin D in the blood is as long as about 2 to 3 weeks, the concentration of 25-hydroxyvitamin D in the blood is considered to be the indicator reflecting the concentration of vitamin D in the body.

25-Hydroxyvitamin D in the blood is taken into cells by endocytosis mediated by megalin receptor. 25-Hydroxyvitamin D transferred to the renal tubules is hydroxylated and converted to the active form of 1,25-dihydroxyvitamin D. The 1,25-dihydroxyvitamin D released into the blood again binds to the vitamin D receptor in the target cell and functions as the transcription factor that controls the expression of various types of genes related to calcium transport and utilization. The half-life of 1,25-dihydroxyvitamin D in the blood is as short as about 15 hours, and the concentration of 1,25-dihydroxyvitamin D in the blood is strictly controlled by parathyroid hormone, calcium, and phosphate. Therefore, it is believed that the concentration of 1,25-dihydroxyvitamin D in the blood will not change unless there is an extreme vitamin D deficiency or excess.

Vitamin D plays an essential role in controlling calcium and phosphate concentrations in the body. Vitamin D mainly affects intestinal cells and osteocytes, where it helps to control calcium uptake in the former and skeletal formation and maintenance in the latter. Also, it is known that vitamin D is related to proliferation and differentiation of the cell and immune system. Vitamin D deficiency or excess has various consequences for the body. In particular, it has been pointed out that vitamin D deficiency may lead to serious diseases such as rickets, osteomalacia, osteoporosis, chronic renal failure, hyperparathyroidism, and psoriasis.

As the criterion for the insufficiency or deficiency of vitamin D, 30 ng/ml or more of 25-hydroxyvitamin D concentration in the serum is considered to be the vitamin D sufficient state, 20 ng/ml or more and less than 30 ng/ml is considered to be the vitamin D insufficient state, and less than 20 ng/ml is considered to be the vitamin D deficient state (according to the judgment guideline of vitamin D insufficiency and deficiency by The Japanese Society for Bone and Mineral Research and The Japan Endocrine Society). The number of patients with vitamin D deficiency is currently estimated to be one billion worldwide. Early detection of vitamin D deficiency is particularly helpful in modern society, where people tend to avoid direct sunlight. In Japan, the electro chemiluminescence immunoassay (ECLIA), the chemiluminescent enzyme immunoassay (CLEIA), and the chemiluminescent immunoassay (CLIA) of 25-hydroxyvitamin D in serum are covered by insurance. These are all immunoassays using an anti-25-hydroxyvitamin D antibody.

For example, Japanese laid-open patent publication No. 2017-40659 discloses an antibody that recognizes 25-hydroxyvitamin D or an antigen-binding fragment thereof and a method for measuring 25-hydroxyvitamin D using the same. In the measurement of 25-hydroxyvitamin D using the anti 25-hydroxyvitamin D antibody, the detection limit of 25-hydroxyvitamin D is less than 3.0 ng/ml and the sensitivity is high, which is useful for the early diagnosis of vitamin D deficiency. However, the immunoassays are time-consuming and expensive.

Therefore, a method for measuring 25-hydroxyvitamin D itself is desired instead of the immunoassay. For example, U.S. Pat. No. 5,981,779 discloses a method for measuring 25-hydroxyvitamin D by competitive binding to vitamin D binding protein using 25-hydroxyvitamin D labeled with biotin or fluorescein. Japanese laid-open patent publication No. 2009-540275 discloses a method for measuring 25-hydroxyvitamin D using high performance liquid chromatography (HPLC). Japanese laid-open patent publication No. 2018-81023 discloses a method for measuring 25-hydroxyvitamin D using a liquid chromatography tandem mass spectrometry (LC/MS/MS)

### SUMMARY

However, the above-mentioned method for measuring 25-hydroxyvitamin D has various drawbacks including long measurement time, measurement error, large cost, sample volume, and difficult-to-handle reagents, and is not optimal for clinical examination. Therefore, there is a need for a method for measuring the concentration of the 25-hydroxyvitamin D that is less laborious, time-consuming, and costly.

One of the objects of the present invention is to provide a novel quantification method for measuring a concentration of 25-hydroxyvitamin D, which is vitamin D derivative, an enzyme for quantification, a composition for quantification, a kit for quantification, an electrode, a sensor chip, and a sensor.

According to an embodiment of the present invention, a quantitation method of vitamin D derivative is provided including adding an oxidoreductase to a sample.

The quantitation method of vitamin D derivative may further include reducing a mediator by adding the oxidoreductase, and reacting the reduced mediator with a reagent to determine a concentration of the vitamin D derivative.

The oxidoreductase may be an oxidase, and hydrogen peroxide produced or oxygen consumed by adding the oxidase may be quantified to determine a concentration of the vitamin D derivative.

The oxidoreductase may be an oxidase, and hydrogen peroxide produced by adding the oxidase may be reacted with a reagent to determine a concentration of the vitamin D derivative.

According to an embodiment of the present invention, an oxidoreductase used in the quantitation method of vitamin D derivative is provided.

The oxidoreductase may be an oxidoreductase belonged to EC No. 1.1.

The oxidoreductase may be an oxidase belonged to EC No. 1.1.3.

According to an embodiment of the present invention, a composition for quantification of vitamin D derivative is provided including the oxidoreductase.

The composition for quantification of vitamin D derivative may further include a mediator to be reduced by adding the oxidoreductase, and a reagent to be reacted with the reduced mediator.

The oxidoreductase may be an oxidase and the composition may additionally include a reagent reacting with hydrogen peroxide produced by adding the oxidase.

According to an embodiment of the present invention, a kit for quantification of vitamin D derivative is provided including the oxidoreductase, a mediator reduced by adding an oxidoreductase, and a reagent reacting with the reduced mediator.

According to an embodiment of the present invention, a kit for quantification of vitamin D derivative is provided including the oxidoreductase and a reagent reacting with hydrogen peroxide.

According to an embodiment of the present invention, an electrode is provided including the oxidoreductase.

According to an embodiment of the present invention, a sensor chip is provided including the electrode as a working electrode.

According to an embodiment of the present invention, a sensor is provided including the sensor chip.

### BRIEF DESCRIPTION OF DRAWINGS

FIG. 1A is a schematic diagram of a sensor chip 10 according to an embodiment of the present invention.

FIG. 1B is a schematic diagram showing a member constituting the sensor chip 10 according to an embodiment of the present invention.

FIG. 1C is a schematic diagram showing a member constituting the sensor chip 10 according to an embodiment of the present invention.

FIG. 1D is a schematic diagram showing a member constituting the sensor chip 10 according to an embodiment of the present invention.

FIG. 2A is a schematic diagram of a sensor 100 according to an embodiment of the present invention.

FIG. 2B is a block diagram of the sensor 100 according to an embodiment of the present invention.

FIG. 3 is a graph showing a relationship between a concentration of vitamin D derivative and absorbance (A₅₅₅) according to an example of the present invention.

FIG. 4 is a graph showing a relationship between a concentration of vitamin D derivative and absorbance (A₅₅₅) according to an example of the present invention.

FIG. 5 is a graph showing a relationship between a concentration of vitamin D derivative and an amount of change in absorbance (A₆₀₀) according to an example of the present invention.

### DESCRIPTION OF EMBODIMENTS

Hereinafter, a novel quantification method for measuring a concentration of vitamin D derivative, an enzyme for quantification, a composition for quantification, a kit for quantification, an electrode, a sensor chip, and a sensor according to the present invention will be described. However, a novel quantification method for measuring a concentration of vitamin D derivative, an enzyme for quantification, a composition for quantification, a kit for quantification, an electrode, a sensor chip, and a sensor according to the present invention should not be construed as being limited to the description of the following embodiments and examples.

In one embodiment, the oxidoreductase used in the present invention is an oxidoreductase that acts on vitamin D derivative as a substrate. Enzymes capable of directly oxidizing or reducing vitamin D derivative have not been identified by the time of the present application. As a result of the examination by the inventors, it was found for the first time that a cholesterol oxidase (ChoF, UniprotKB Entry name Q56DL0-9MICC) derived from the F2 strain of the genus Arthrobacter (Arthrobacter sp.) acts on 25-hydroxyvitamin D₃, which is vitamin D derivative. In addition, glucose-methanol-choline family oxidoreductase (HeGMCOR, NCBI Reference Sequence: WP_094565544.1) derived from the meg3 strain of the genus Herbaspirillum (Herbaspirillum sp.) was found to act on 25-hydroxyvitamin D₃, which is vitamin D derivative. In this specification, although a cholesterol oxidase derived from the F2 strain of the genus Arthrobacter is shown and described as an example of the oxidoreductase, the present invention is not limited thereto, and may include those having certain level of reactivity with vitamin D derivative.

For example, among oxidoreductases belonging to EC No. 1.1, an enzyme that recognizes vitamin D derivative as a substrate and has the vitamin D derivative oxidoreductase activity can be used as the oxidoreductase. For example, an oxidase belonging to EC No. 1.1, recognizing vitamin D derivative as a substrate, and having vitamin D derivative oxidase activity can be used. For example, an oxidase belonging to EC No. 1.1.3, recognizing vitamin D derivative as a substrate, and having vitamin D derivative oxidase activity can be used. For example, a cholesterol oxidase belonging to EC No. 1.1.3.6, recognizing vitamin D derivative as a substrate, and having vitamin D derivative oxidase activity can be used. For example, an oxidoreductase belonging to EC No. 1.1, recognizing vitamin D derivative as a substrate, and having vitamin D derivative dehydrogenase activity can be used. For example, an oxidase belonging to EC No. 1.1.3, recognizing vitamin D derivative as a substrate, and having vitamin D derivative dehydrogenase activity can be used. For example, a cholesterol oxidase belonging to EC No. 1.1.3.6, recognizing vitamin D derivative as a substrate, and having vitamin D derivative dehydrogenase activity can be used.

In one embodiment, the oxidoreductase may be an oxidoreductase produced by a naturally occurring microorganism or an oxidoreductase produced by a transformed microorganism. From the viewpoint of efficient mass expression of the enzyme, the enzyme can be efficiently expressed in large quantities by using the transformed microorganism.

In one embodiment, the oxidoreductase may be a multimer or a monomer. For example, when only a certain subunit (monomer) among several subunits constituting the oxidoreductase, which is a multimer, catalyzes a dehydrogenation reaction in which hydrogen is taken from a substrate to a hydrogen acceptor, the oxidoreductase used in the present invention may be a multimer or the subunit (monomer). Also, the oxidoreductase may be a partial structure of an enzyme as long as it has vitamin D derivative oxidoreductase activity.

As described above, the inventors have found for the first time that the cholesterol oxidase derived from the F2 strain of the genus Arthrobacter acts on 25-hydroxyvitamin D₃, which is vitamin D derivative. In one embodiment, the oxidoreductase of the present invention includes an oxidoreductase derived from the F2 strain of the genus Arthrobacter, but also an oxidoreductase derived from microorganisms classified as the class Actinobacteria, and an oxidoreductase derived from a microorganism classified as the genus Arthrobacter. In one embodiment, the oxidoreductase of the present invention includes an oxidoreductase derived from microorganisms classified as the genus Herbaspirillum or the genus Pedobacter. In one embodiment, the oxidoreductase of the present invention includes an oxidoreductase derived from a microorganism classified as the genus Corynebacterium, the genus Rhodococcus, the genus Brevibacterium, the genus Nocardia, the genus Vitiosangium, the genus Dietzia, the genus Tomitella, the genus Actinomadura, the genus Actinoallomurus. In one embodiment, the oxidoreductase of the present invention includes an oxidoreductase derived from microorganisms classified as the genus Amycolatopsis, the genus Actinoplanes, the genus Krasilnikovia, the genus Couchioplanes, the genus Streptosporangium, the genus Nonomuraea, the genus Streptacidiphilus, the genus Nocardioides, the genus Alloactinosynnema, the genus Microbispora, the genus Actinocrispum, the genus Kutzneria, the genus Lentzea, the genus Kibdelosporangium, the genus Catenulispora, the genus Planomonospora, the genus Dyella, the genus Marmoricola, the genus Actinosynnema, the genus Prauserella, and the genus Yuhushiella. An oxidoreductase having high sequence identity (e.g., 50% or more, 51% or more, 52% or more, 53% or more, 54% or more, 55% or more, 56% or more, 57% or more, 58% or more, 59% or more, 60% or more, 61% or more, 62% or more, 63% or more, 64% or more, 65% or more, 66% or more, 67% or more, 68% or more, 69% or more, 70% or more, 71% or more, 72% or more, 73% or more, 74% or more, 75% or more, 76% or more, 77% or more, 78% or more, 79% or more, 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more, e.g., 99% or more) with respect to the amino acid sequence of the oxidoreductase described in SEQ ID NO: 1, and oxidoreductase having an amino acid sequence in which 1 or more amino acids are modified or mutated, deleted, substituted, added and/or inserted in the amino acid sequence of SEQ NO: 1. In addition, the oxidoreductase can be screened by culturing the microorganism of the F2 strain of the genus Arthrobacter under a predetermined condition (for example, see Journal of the Japanese Society for Bacteriology, 18 (1), 1963), mixing an oxidase reaction reagent or a dehydrogenase reaction reagent (described in detail later) containing vitamin D derivative with an extract obtained by crushing the bacterial cells, and confirming the presence or absence of reactivity with the reagent.

In one embodiment, the present invention provides DNA encoding oxidoreductase. In one embodiment, the present invention provides DNA encoding amino acid sequence shown in SEQ ID NO: 1 or DNA having the base sequence shown in SEQ ID NO: 2. In one embodiment, the present invention provides DNA including the base sequence having 40% or more, 41% or more, 42% or more, 43% or more, 44% or more, 45% or more, 46% or more, 47% or more, 48% or more, 49% or more, 50% or more, 51% or more, 52% or more, 53% or more, 54% or more, 55% or more, 56% or more, 57% or more, 58% or more, 59% or more, 60% or more, 61% or more, 62% or more, 63% or more, 64% or more, 65% or more, 66% or more, 67% or more, 68% or more, 69% or more, 70% or more, 71% or more, 72% or more, 73% or more, 74% or more, 75% or more, 76% or more, 77% or more, 78% or more, 79% or more, 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more, or 99% or more of sequence identity with the base sequence shown in SEQ ID NO: 2, and encoding a protein having oxidoreductase activity.

In one embodiment, the oxidoreductase of the present invention may be the oxidoreductase derived from the F2 strain of the genus Arthrobacter or the oxidoreductase produced by Escherichia coli transformed with a plasmid containing the oxidoreductase gene derived from the F2 strain of the genus Arthrobacter. The oxidoreductase can be efficiently expressed in large quantities by using the E. coli transformed with the plasmid containing the oxidoreductase gene derived from the F2 strain of the genus Arthrobacter.

The inventors have further found for the first time that the glucose-methanol-choline family oxidoreductase (GMC family oxidoreductase) derived from the meg3 strain of the genus Herbaspirillum acts on the 25-hydroxyvitamin D₃, which is vitamin D derivative. In an embodiment, the oxidoreductase of the present invention includes the GMC family oxidoreductase derived from the meg3 strain of the genus Herbaspirillum, but also the GMC family oxidoreductase derived from a microorganism classified as the genus Pedobacter or the GMC family oxidoreductase derived from a microorganism classified as the genus Pedobacter cryoconitis. In one embodiment, the oxidoreductase of the present invention includes the oxidoreductases derived from microorganisms classified as the genus Rhodococcus, the genus Brevibacterium, the genus Arthrobacter, the genus Dietzia, the genus Actinoallomurus, the genus Nocardia, the genus Chryseobacterium, the genus Streptomyces, the genus Flavobacterium, the genus Kaistella, the genus Ornithobacterium, the genus Phychrobacter, the genus Riemerella, the genus Goodfellowiella, the genus Lentzea, the genus Microscilla, the genus Hymenobacter, the genus Amycolatopsis, the genus Prauserella, the genus Kribbella, the genus Actinobacteria, the genus Soonwooa, the genus Elizabethkingia, the genus Tamaricihabitans, the genus Bizionia, the genus Saccharopolyspora, the genus Weeksella, the genus Harbihabitans, the genus Thalassolituus, the genus Vitiosangium, the genus Streptacidiphilus, the genus Aquabacterium, the genus Kutzneria, the genus Saccharothrix, and the genus Actinokineospora. An oxidoreductase having high sequence identity (e.g., 50% or more, 51% or more, 52% or more, 53% or more, 54% or more, 55% or more, 56% or more, 57% or more, 58% or more, 59% or more, 60% or more, 61% or more, 62% or more, 63% or more, 64% or more, 65% or more, 66% or more, 67% or more, 68% or more, 69% or more, 70% or more, 71% or more, 72% or more, 73% or more, 74% or more, 75% or more, 76% or more, 77% or more, 78% or more, 79% or more, 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more, e.g., 99% or more) with respect to the amino acid sequence of the oxidoreductase described in SEQ ID NO: 12, and an oxidoreductase having an amino acid sequence in which 1 or more amino acids are modified or mutated, deleted, substituted, added and/or inserted in the amino acid sequence of SEQ NO: 12. In addition, the oxidoreductase can be screened by culturing a microorganism of the meg3 strain of the genus Herbaspirillum under a predetermined condition (for example, see Journal of the Japanese Society for Bacteriology, (1), 1963), mixing an oxidase reaction reagent or a dehydrogenase reaction reagent (described in detail later) containing vitamin D derivative with an extract obtained by crushing the bacterial cells, and confirming the presence or absence of reactivity with the reagent.

In one embodiment, the present invention provides DNA encoding oxidoreductase. In one embodiment, the present invention provides DNA encoding amino acid sequence shown in SEQ ID NO: 12 or DNA having the base sequence shown in SEQ ID NO: 9. In one embodiment, the present invention provides DNA including the base sequence having 40% or more, 41% or more, 42% or more, 43% or more, 44% or more, 45% or more, 46% or more, 47% or more, 48% or more, 49% or more, 50% or more, 51% or more, 52% or more, 53% or more, 54% or more, 55% or more, 56% or more, 57% or more, 58% or more, 59% or more, 60% or more, 61% or more, 62% or more, 63% or more, 64% or more, 65% or more, 66% or more, 67% or more, 68% or more, 69% or more, 70% or more, 71% or more, 72% or more, 73% or more, 74% or more, 75% or more, 76% or more, 77% or more, 78% or more, 79% or more, 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more, or 99% or more of sequence identity with the base sequence shown in SEQ ID NO: 9, and encoding a protein having oxidoreductase activity is provided.

In one embodiment, the oxidoreductase of the present invention may be the oxidoreductase derived from the meg3 strain of the genus Herbaspirillum or the oxidoreductase produced by E. coli transformed with a plasmid containing an oxidoreductase gene derived from the meg3 strain of the genus Herbaspirillum. The oxidoreductase can be efficiently expressed in large quantities by using E. coli transformed with the plasmid containing the oxidoreductase gene derived from the meg3 strain of the genus Herbaspirillum.

In one embodiment, a reaction condition of the oxidoreductase may be any condition as long as it is a condition for acting on vitamin D derivative and efficiently catalyzing an oxidation reaction or a reduction reaction. Generally, an enzyme has an optimum temperature and optimum pH that exhibit the highest activity. Therefore, it is suitable that the reaction condition is near the optimum temperature and the optimum pH. In one embodiment, the reaction condition of the oxidoreductase is comprehensively examined from a suitable condition for a component such as a composition other than an enzyme, for example, a coloring reagent, a mediator, a stabilizer of an enzyme, or a stabilizer of a measurement sample, compatibility with a measurement device, and the like, and a method of quantifying vitamin D derivative under conditions other than an optimum condition of an enzyme alone is also included in the measurement method of the present invention.

### [Vector]

As a vector that can be used in the present invention, for example, any vector known to those skilled in the art such as bacteriophage, cosmid, and the like can be used. Specifically, for example, pUC18 (manufactured by Takara Bio Inc.), pBluescriptII SK+ (manufactured by STRATAGENE), pET-22b(+) (manufactured by Merck), pKK223-3 (manufactured by addgene) and the like are preferred.

### [Construction of Expression Plasmid]

The plasmid for expressing an oxidoreductase according to the present invention is obtained by a commonly used method. For example, DNA is extracted from a microorganism producing an oxidoreductase according to the present invention to create a DNA library. A DNA fragment encoding the oxidoreductase according to the present invention is identified and isolated from the constructed DNA library. The DNA fragment is amplified by a polymerase chain reaction (PCR) with a complementary primer in which the isolated DNA fragment is used as a template to clone a gene encoding the oxidoreductase according to the present invention. The amplified DNA fragment is ligated into a vector to obtain a plasmid having DNA fragment encoding the oxidoreductase according to the present invention.

Alternatively, the DNA fragment encoding the oxidoreductase according to the present invention is chemically synthesized, and the DNA fragment is ligated into the vector to obtain the plasmid having DNA encoding the oxidoreductase according to the present invention.

### [Mutation Treatment of Oxidoreductase Gene]

Mutational treatment of the oxidoreductase gene can be performed in any known method, depending on the intended mutant form. That is, a method using a genetic engineering method, a protein engineering method, or the like can be widely used.

Generally, a method known as Site-Specific Mutagenesis can be used as the method utilizing the protein engineering method. For example, Kramer method (Nucleic Acids Res., 12, 9441 (1984): Method Enzymol., 154, 350 (1987): Gene, 37, 73 (1985)), Eckstein method (Nucleic Acids Res., 13, 8749 (1985): Nucleic Acids Res., 13, 8765 (1985): Nucleic Acids Res, 14, 9679 (1986)), Kunkel method (Proc. Natl. Acid. Sci. U.S.A., 82, 488 (1985): Method Enzymol., 154, 367 (1987)), and the like can be exemplified. Specific methods for converting sequences in DNA include, for example, the use of commercially available kits (Transformer Mutagenesis Kit; manufactured by Clonetech Laboratories, Inc., EXOIII/Mung Bean Deletion Kit; manufactured by Stratagene Corporation, Quick Change Site Directed Mutagenesis Kit; manufactured by Stratagene Corporation, and the like).

A method known as the common PCR method (Polymerase Chain Reaction) can be used (Technique, 1, 11(1989)). In addition to the above genetic modification method, a desired modified oxidoreductase gene can be directly synthesized by an organic synthesis method or an enzyme synthesis method.

Determination or confirmation of the DNA sequence of the oxidoreductase gene obtained by the above methods can be performed by using, for example, Applied Biosystems 3730xl DNA analyzer (manufactured by Thermo Fisher Scientific).

### [Transformation and Transduction]

The oxidoreductase gene obtained as described above can be incorporated into a vector such as bacteriophage, cosmid, or plasmid used for transformation of prokaryotic or eukaryotic cells by a conventional method, and a host corresponding to each vector can be transformed or transduced by a conventional method. For example, the obtained recombinant DNA can be used to transform or transduce any host, e.g., microorganisms belonging to the genus Escherichia, in particular, E. coli K-12 strains, preferably E. coli JM109 strains, E. coli DH5a strains (both produced by Takara Bio, Inc.), E. coli B strains, preferably E. coli BL21 strains (produced by NIPPON GENE CO., LTD) or the like, to obtain each strain.

Further, for example, an example of a eukaryotic host cell is yeast. Microorganisms classified as yeast include, for example, yeast belonging to the genus Zygosaccharomyces, the genus Saccharomyces, the genus Pichia, and the genus Candida. An insertion gene may include a marker gene to allow the selection of transformed cells. The marker gene includes, for example, genes that complement the auxotrophy of the host, such as URA3 and TRP1. In addition, the insertion gene preferably contains a promoter or other control sequence capable of expressing the gene of the present invention in a host cell, (e.g., secretion signal sequence, enhancer sequence, terminator sequence, polyadenylation sequence, etc.). Specific examples of the promoter include GAL1 promoter, ADH1 promoter, and the like. Although a known method, for example, a method using lithium acetate (Methods Mol. Cell. Biol., 5, 255-269 (1995)), electroporation (J Microbiol Methods 55 (2003) 481-484), or the like, can be suitably used as a transformation method to yeast, the present invention is not limited thereto, and transformation can be performed using various optional methods including a spheroplast method, a glass beads method, and the like.

Other examples of eukaryotic host cells include, for example, filamentous fungi such as the genus Aspergillus and the genus Trichoderma. A method of producing a transformant of the filamentous fungi is not particularly limited, and for example, includes a method of inserting into the host filamentous fungi in a manner in which the gene encoding the oxidoreductase is expressed according to a conventional method. Specifically, a transformant overexpressing the gene encoding the oxidoreductase is obtained by producing a DNA construct in which the gene encoding the oxidoreductase is inserted between an expression-inducing promoter and terminator, then transforming the host filamentous fungi with the DNA construct containing the gene encoding the oxidoreductase of the present invention. In this specification, the DNA fragment consisting of the expression-inducing promoter—the gene encoding the oxidoreductase—the terminator and the recombinant vector containing the DNA fragment produced for transforming a host filamentous fungi are collectively referred to as a DNA construct.

The method of inserting the gene encoding the oxidoreductase into the host filamentous fungi in such manner that it is expressed is not particularly limited, and for example, the method includes a method of inserting the gene directly into the chromosome of the host organism by utilizing homologous recombination, and a method of introducing the gene into the host filamentous fungi by ligating the gene into a plasmid vector, and the like.

In a method utilizing homologous recombination, the DNA construct can be ligated between sequences homologous to the upstream region and the downstream region of the recombination site on the chromosome and inserted into the genome of the host filamentous fungi. Transformants by self-cloning can be obtained by overexpressing in the host filamentous fungi under the control of the high expression promoter of the host filamentous fungi itself. The high expression promoter is not particularly limited, and examples thereof include a promoter region of a TEF1 gene (tef1), which is a translational elongation factor, a promoter region of an α-amylase gene (amy), and an alkaline protease gene (alp) promoter region.

In a method utilizing the vector, the DNA construct can be incorporated into the plasmid vector used in the transformation of filamentous fungi in a conventional method, and the corresponding host filamentous fungi can be transformed by a conventional method.

Such suitable vector-host systems are not particularly limited as long as they are the system capable of producing the oxidoreductase in the host filamentous fungi, and examples thereof include a system of pUC19 and filamentous fungi, and a system of pSTA14 (Mol. Gen. Genet. 218, 99-104, 1989) and filamentous fungi.

Although the DNA construct is preferably used by introducing it into the chromosome of the host filamentous fungi, it can also be used by incorporating the DNA construct into an autonomously replicated vector without introducing it into the chromosome (Ozeki et al. Biosci. Biotechnol. Biochem 59, 1133 (1995)).

The DNA construct may include a marker gene to allow the selection of transformed cells. The marker gene is not particularly limited, and examples of the marker gene include the genes that complement auxotrophy of the host, such as pyrG, niaD, adeA; and the drug resistance genes against a drug, such as pyrithiamine, hygromycin B, or oligomycin. It is also preferred that the DNA construct contains a promoter, a terminator, or other regulatory sequences (e.g., an enhancer, a polyadenylation sequence, and the like) that allow for overexpression of the gene encoding the oxidoreductase of the present invention in the host cell. The promotor includes, but are not limited to, an appropriate expression-inducing promoter and a constitutive promoter, such as a tef1 promoter, an alp-promoter, an amy-promoter, and the like. The terminator is also not particularly limited, and examples thereof include an alp terminator, an amy terminator, and a tef1 terminator.

In the DNA construct, the expression control sequence of the gene encoding the oxidoreductase is not necessarily required when the DNA fragment containing the gene encoding the oxidoreductase to be inserted, contains a sequence having the expression control function. When transformation is performed by a co-transformation method, the DNA construct may not have a marker gene in some cases.

One embodiment of the DNA construct is a DNA construct in which, for example, a tef1 promoter, a gene encoding the oxidoreductase, an alp terminator, and a pyrG marker gene are ligated to an In-Fusion Cloning Site at a multicloning site of pUC19.

As a method for transforming into filamentous fungi, a method known to those skilled in the art can be appropriately selected, and for example, a protoplast PEG method using polyethylene glycol and calcium chloride (see, for example, Mol. Gen. Genet. 218, 99-104, 1989, Japanese laid-open patent publication No. 2007-222055, and the like) can be used after preparing a protoplast of a host filamentous fungi. An appropriate medium is used for the regeneration of the transformed filamentous fungi depending on the host filamentous fungi and the transformation marker gene to be used. For example, when Aspergillus sojae is used as the host filamentous fungi and a pyrG gene is used as the transformation marker gene, regeneration of the transformed filamentous fungi can be performed, for example, in a Czapek-Dox minimal medium (manufactured by Difco Laboratories) containing 0.5% agar and 1.2 M sorbitol.

### [Identity or Similarity of Amino Acid Sequence]

The identity or similarity of the amino acid sequence can be calculated by a program such as maximum matching or search homology of GENETYX Ver. 11 or Ver. 14 (manufactured by Genetyx Corporation), or maximum matching or multiple alignment of DNASIS Pro (manufactured by Hitachi Solutions, Ltd.). When the amino acid sequences of 2 or more oxidoreductases are aligned in order to calculate the amino acid sequence identity, a position of the amino acid which is identical in the 2 or more oxidoreductases can be examined. An identical region in the amino acid sequence can be determined based on such information.

Also, a position of the amino acid which is similar in the 2 or more oxidoreductases can be examined. For example, CLUSTALW can be used to align a plurality of amino acid sequences, in this case, Blosum62 is used as an algorithm, and amino acids judged to be similar when a plurality of amino acid sequences are aligned may be referred to as similar amino acids. In the mutant of the present invention, the amino acid substitution may occur by substitution between such similar amino acids. By such alignment, it is possible to investigate the region having identical amino acid sequences and the position occupied by the similar amino acids. Based on this information, a homology region (conserved region) in the amino acid sequence can be determined.

### [Method for Enzyme Preparation]

Hereinafter, a method for preparing an oxidoreductase according to the present invention will be described.

A strain such as E. coli is transformed with the plasmid having DNA encoding an oxidoreductase according to the present invention to obtain a strain such as E. coli having DNA encoding the oxidoreductase according to the present invention.

### [Recombinant Expression of Enzyme]

The strain such as E. coli having the DNA encoding the oxidoreductase of the present invention is cultured in a culture medium. When culturing a microbial host cell, it may be carried out by aeration stirring deep culture, shaking culture, static culture, or the like, at a culture temperature of 10° C. to 42° C., preferably at a culture temperature of about 25° C., for several hours to several days, and more preferably at a culture temperature of about 25° C. for 1 to 7 days. Any conventional medium in which filamentous fungi are cultured, i.e., a synthetic medium or a natural medium, can be used as long as it contains an appropriate proportion of a carbon source, a nitrogen source, an inorganic substance, or other nutrients. Further, as the medium for culturing the microbial host cell, for example, a medium in which one or more kinds of inorganic salts such as sodium chloride, monopotassium phosphate, dipotassium phosphate, magnesium sulfate, magnesium chloride, ferric chloride, ferric sulfate, or manganese sulfate are added to one or more kinds of nitrogen sources such as yeast extract, tryptone, peptone, meat extract, corn steep liquor, or leaching solution of soybean or wheat bran, and if necessary, a sugar raw material, vitamins, and the like are appropriately added is used.

Culture conditions of filamentous fungi commonly known by those skilled in the art may be adopted as culture conditions, and for example, an initial pH of the medium may be adjusted to 5 to 10, and the culture temperature may be appropriately set to 20° C. to 40° C., the culture time may be set for several hours to several days, preferably for 1 to 7 days, more preferably for 2 to 5 days, and the like. The culture means is not particularly limited, and aeration stirring deep culture, shaking culture, static culture, and the like can be adopted, but it is preferred to culture under conditions such that dissolved oxygen becomes sufficient. For example, an example of a medium and a culturing condition for culturing the Aspergillus microorganism includes a shaking culture at 30° C. at 160 rpm for 3 to 5 days using DPY medium.

After completion of the culture, the oxidoreductase of the present invention is collected from the culture. For this purpose, conventional known enzyme collection means may be used. For example, the culture medium supernatant fraction can be collected, or the bacterial cell can be subjected to ultrasonic pulverization treatment, grinding treatment, or the like by a conventional method, or the enzyme can be extracted using a lytic enzyme such as lysozyme or yatalase, or the bacterial cell can be shaken or leave to lyse in the presence of toluene or the like, and the enzyme can be discharged to the outside of the bacterial cell. Then, the solution is filtered, centrifuged, or the like to remove a solid portion, and if necessary, a nucleic acid is removed by streptomycin sulfate, protamine sulfate, manganese sulfate, or the like, and then ammonium sulfate, alcohol, acetone, or the like is added thereto to fractionate, and a precipitate is collected to obtain a crude enzyme of the oxidoreductase of the present invention.

### [Purification of Enzyme]

The method for purifying an enzyme may be any method as long as it is capable of purifying an enzyme from a crude enzyme solution. For example, a purified oxidoreductase enzyme preparation of the present invention can be obtained by appropriately selecting or combining a gel filtration method using Sephadex, Ultrogel or Biogel or the like, an adsorption elution method using an ion exchanger, an electrophoresis method using a polyacrylamide gel or the like, an adsorption elution method using hydroxyapatite, a sedimentation method such as a sucrose density gradient centrifugation method, an affinity chromatography method, a fractionation method using a molecular sieve membrane or a hollow fiber membrane or the like, or the like.

### [Enzyme Activity Measurement]

The method for measuring the activity of the enzyme may be any method as long as it directly or indirectly measures a product of a redox reaction catalyzed by the enzyme. For example, a reduced product is produced by catalyzing a redox reaction by an enzyme, and a current value generated by passing electrons from the reduced product to an electrode is measured, so that enzyme activity can be measured. In addition, oxygen is consumed by the enzyme catalyzing the redox reaction, and the enzyme activity can be measured from the consumption of oxygen by an electrochemical method using an oxygen electrode, for example. Suitably, the enzyme activity can be measured by reacting the reduced product by the redox reaction catalyzed by the enzyme with a reagent containing an absorbing substance reacting with the reduced product (hereinafter, an “absorbing reagent”) and performing absorbance measurement.

### [Composition Containing Oxidoreductase and Kit for Quantification of Vitamin D Derivative]

A quantification method of vitamin D derivative utilizing the oxidoreductase according to the present invention may be carried out by providing a composition containing the oxidoreductase and a product reaction reagent, or may be carried out by combining the oxidoreductase and a commercially available product reaction reagent.

The quantification method of vitamin D derivative, the oxidoreductase for quantification, the composition for quantification, and the kit for quantification may provide a novel quantification method of vitamin D derivative that is an indicator of diseases associated with vitamin D deficiency, an enzyme for quantification, a composition for quantification, a kit for quantification, an electrode, a sensor chip, and a sensor by containing an oxidoreductase.

### [Sensor Chip and Electrode]

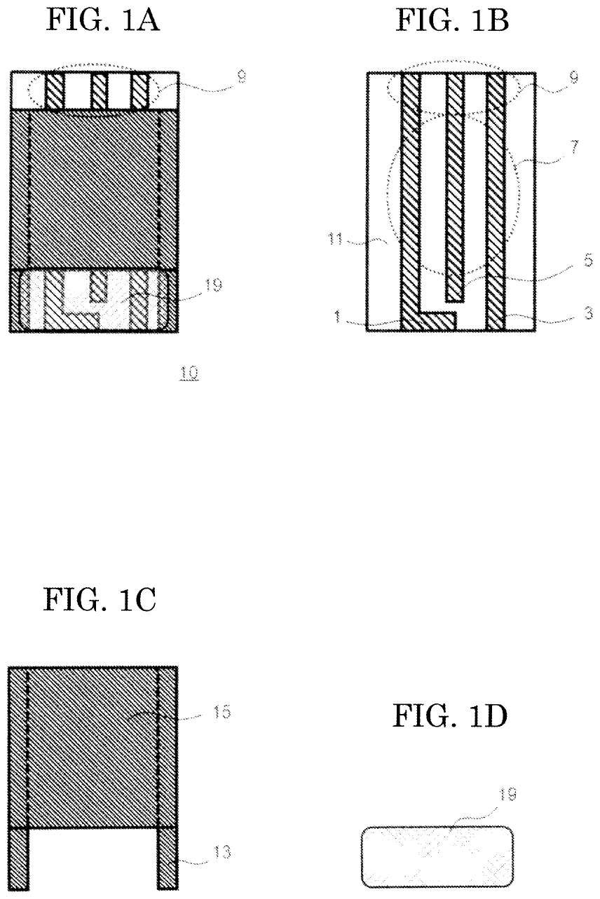

FIG. 1A is a schematic diagram of a sensor chip 10 according to an embodiment of the present invention, and FIG. 1B to FIG. 1D are schematic diagrams showing members constituting the sensor chip 10. The sensor chip 10 includes two or more electrodes arranged on a substrate 11. The substrate 11 is made of an insulating material. In FIG. 1A and FIG. 1B, as an example, a working electrode 1, a counter electrode 3, and a reference electrode 5 are arranged on the substrate 11. Each electrode is electrically connected to a wiring portion 7, and the wiring portion 7 is electrically connected to a terminal 9 located on the opposite side of the electrode. The working electrode 1, the counter electrode 3, and the reference electrode 5 are arranged apart from each other. The working electrode 1, the counter electrode 3, and the reference electrode 5 are preferably formed integrally with the wiring portion 7 and the terminal 9. Further, the counter electrode 3 and the reference electrode 5 may be integral.

As shown in FIG. 1A and FIG. 1C, a spacer 13 is arranged on an end of the substrate 11 which is parallel to the wiring portion 7, and a cover 15 which covers the working electrode 1, the counter electrode 3, the reference electrode 5, and the spacer 13 is arranged. The spacer 13 and the cover 15 are made of an insulating material. The spacer 13 preferably has a thickness substantially equal to that of the working electrode 1, the counter electrode 3, and the reference electrode 5, and is in close contact with the working electrode 1, the counter electrode 3, and the reference electrode 5. The spacer 13 and the cover 15 may be integrally formed. The cover 15 is a protective layer which prevents the wiring portion 7 from deteriorated by being exposed to the outside air and short-circuiting due to the penetration of the measurement sample.

In an embodiment, the oxidoreductase of the present invention may be applied, adsorbed, or immobilized on the electrode. Preferably, the oxidoreductase of the present invention is applied, adsorbed, or immobilized on the working electrode 1. In another embodiment, the mediator together with the oxidoreductase may also be applied, adsorbed, or immobilized on the electrode. The oxidoreductase, or the oxidoreductase and the mediator may be included in a reaction layer 19 arranged on the working electrode 1, the counter electrode 3, and the reference electrode 5. As the electrode, a carbon electrode, a metal electrode such as platinum, gold, silver, nickel, or palladium can be used. In the case of carbon electrodes, examples of the material include pyrolytic graphite carbon (PG), glassy carbon (GC), carbon paste and plastic foamed carbon (PFC). A measurement system may be a two-electrode system or a three-electrode system, for example, enzymes may be immobilized on the working electrode. Examples of the reference electrode include a standard hydrogen electrode, a reversible hydrogen electrode, a silver-silver chloride electrode (Ag/AgCl), a palladium-hydrogen electrode, and a saturated calomel electrode, and the Ag/AgCl is preferably used from the viewpoint of stability and reproducibility.

The enzymes can be immobilized on the electrode by crosslinking, coating with a dialysis membrane, encapsulation in a polymer matrix, use of a photocrosslinkable polymer, use of a conductive polymer, use of an oxidation/reduction polymer, and the like. The enzymes may also be immobilized in a polymer or adsorbed onto the electrode together with a mediator, or these techniques may be combined.

The mediator (also referred to as an artificial electron mediator, an artificial electron acceptor or an electron mediator) used in the composition, kit, electrode, or sensor chip of the present invention is not particularly limited as long as it can receive electrons from an oxidoreductase. Examples of the mediators include quinones, phenazines, viologens, cytochromes, phenoxazines, phenothiazines, ferricyanides, e.g., potassium ferricyanide, ferredoxins, ferrocene, osmium complexes and derivatives thereof, and the like, and examples of the phenazine compounds include, but are not limited to, 5-Methylphenazinium methosulfate (PMS) and methoxy PMS.

The oxidoreductase of the present invention can be applied to various electrochemical measurement methods by using a potentiostat, a galvanostat, or the like. The electrochemical measurement includes various techniques such as amperometry, potentiometry, and coulometry. For example, in the case of the amperometry method, the concentration of vitamin D derivative in a sample can be calculated by measuring a current value generated by applying +600 mV to +1000 mV (vs. Ag/AgCl) by a hydrogen peroxide electrode to hydrogen peroxide produced when oxidoreductase reacts with vitamin D derivative. For example, a calibration curve can be generated by measuring current values for known concentrations of vitamin D derivative (0, 5, 10, 50 μM) and plotting against the concentrations of vitamin D derivative. The concentration of vitamin D derivative can be obtained from the calibration curve by measuring the current value of the unknown vitamin D derivative concentration. As the hydrogen peroxide electrode, for example, a carbon electrode or a platinum electrode can be used. The amount of hydrogen peroxide can be quantified by measuring the reduction current value generated by applying −400 mV to +100 mV (vs. Ag/AgCl) using an electrode immobilized with a reductase such as a peroxidase or catalase, instead of the hydrogen peroxide electrode, and the value of vitamin D derivative can also be measured.

By, for example, an amperometry method, the concentration of vitamin D derivative in the sample can be calculated by mixing a mediator in a reaction solution, transferring electrons generated when oxidoreductase reacts with vitamin D derivative to an oxidized mediator, generating a reduced mediator, and measuring a current value generated by applying −1000 mV to +500 mV (vs. Ag/AgCl). As the counter electrode, a carbon electrode or a platinum electrode is preferred. For example, a calibration curve can be generated by measuring current values for known concentrations of vitamin D derivative (0, 100, 200, 500 μM) and plotting against the concentrations of vitamin D derivative. The concentration of vitamin D derivative can be obtained from the calibration curve by measuring the current value of the unknown vitamin D derivative.

In addition, printed electrodes (sensor chips) can be used to reduce the amount of solution required for measurement. In this case, the electrodes are preferably formed on a substrate composed of an insulating substrate. Specifically, the electrodes are preferably formed on the substrate by photolithography or printing techniques such as screen printing, gravure printing, and flexographic printing. Further, examples of the material of the insulating substrate include silicon, glass, ceramic, polyvinyl chloride, polyethylene, polypropylene, and polyester, but those having strong resistance to various solvents and chemicals are more preferably used.

### [Vitamin D Derivative Measurement Sensor]

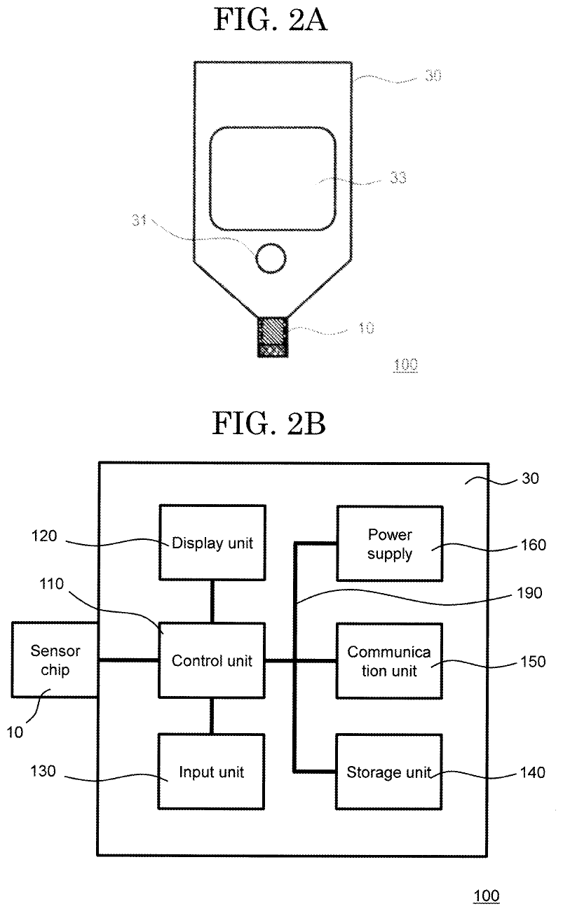

In an embodiment, a vitamin D derivative measurement sensor using an oxidoreductase of the invention is provided. FIG. 2A is a schematic diagram of a sensor 100 according to an embodiment of the present invention. The sensor is a vitamin D derivative measurement device using an oxidoreductase of the present invention and includes the sensor chip containing the oxidoreductase, and a measurement unit. A measurement unit 30 may include, for example, a switch 31 serving as an input unit and a display 33 serving as a display unit. The switch 31 may be used, for example, to control ON/OFF of a power supply of the measurement unit 30, or to control the initiation or interruption of the measurement of the vitamin D derivative by the sensor 100. The display 33 may display, for example, a measured value of vitamin D derivative, and may include a touch panel as an input unit for controlling the measurement unit 30.

FIG. 2B is a block diagram of the sensor 100 according to an embodiment of the present invention. The sensor 100 may include, for example, a control unit 110, a display unit 120, an input unit 130, a storage unit 140, a communication unit 150, and a power supply 160 in the measurement unit 30, which may be electrically connected to each other by a wiring 190. Further, a terminal of the sensor chip 10 to be described later and a terminal of the measurement unit 30 are electrically connected, and the current generated at the sensor chip 10 is detected by the control unit 110. The control unit 110 is a control device which controls the sensor 100 and is composed of, for example, a known central processing unit (CPU) and an operation program which controls the sensor 100. The control unit 110 may include a central processing unit and an operating system (OS) and may include application programs or modules for performing vitamin D derivative measurements.

The display unit 120 may include, for example, the known display 33, and may display the measured values of vitamin D derivative, states of the measurement unit 30, and requests for operations to the measurer. The input unit 130 is an input device for the measurer to operate the sensor 100, and may be, for example, switch 31 or a touch panel arranged on the display 33. A plurality of switches 31 may be arranged in the measurement unit 30.

The storage unit 140 consists of a main storage device (memory) and an auxiliary storage device (hard disk) may be arranged externally. The main storage device (memory) may be composed with a read-only memory (ROM) and/or random access memory (RAM). The operation program, operating system, application program, or module is stored in the storage unit 140 and executed by the central processing unit to configure the control unit 110. The measured values and the current values can be stored in the storage unit 140.

The communication unit 150 is a known communication device which connects the sensor 100 or the measurement unit to external devices (such as computers, printers, or networks). The communication unit 150 and external devices are connected by wired or wireless communication. The power supply 160 is also a known power supply device which supplies power to the sensor 100 or the measurement unit 30.

As described above, the quantification method of vitamin D derivative, the oxidoreductase for quantification, the composition for quantification, and the kit for quantification according to the present invention can provide a novel quantification method for quantifying the concentration of vitamin D derivative, a novel enzyme for quantification, a novel composition for quantification, a novel kit for quantification, a novel electrode, a novel sensor chip, and a novel sensor, by containing the oxidoreductase.

### [Quantification Method of Vitamin D Derivative Using Dehydrogenase Activity]

The dehydrogenase used in the present invention is an enzyme which acts on vitamin D derivative as a substrate, oxidizes vitamin D derivative, and passes the withdrawn electron to various mediators. However, by the time of filing the present application, no dehydrogenase acting on vitamin D derivative has been identified. In this specification, an oxidoreductase having dehydrogenase activity is referred to as a dehydrogenase.

In one embodiment, the dehydrogenase may include an oxidoreductase selected from the oxidoreductases described above or having high vitamin D derivative dehydrogenase activity among the oxidoreductases described above. In one embodiment, the dehydrogenase includes cholesterol oxidase derived from the F2 strain of the genus Arthrobacter described above. In one embodiment, the dehydrogenase of the present invention includes oxidoreductases derived from the F2 strain of the genus Arthrobacter, but also oxidoreductases derived from microorganisms classified as the class Actinobacteria, and oxidoreductase derived from microorganisms classified as the genus Arthrobacter. In one embodiment, the dehydrogenase of the present invention includes an oxidoreductase derived from a microorganism classified as the genus Corynebacterium, the genus Rhodococcus, the genus Brevibacterium, the genus Nocardia, the genus Vitiosangium, the genus Dietzia, the genus Tomitella, the genus Actinomadura, the genus Actinoallomurus. In an embodiment, examples of the oxidoreductase of the present invention include the genus Amycolatopsis, the genus Actinoplanes, the genus Krasilnikovia, the genus Couchioplanes, the genus Streptosporangium, the genus Nonomuraea, the genus Streptacidiphilus, the genus Nocardioides, the genus Alloactinosynnema, the genus Microbispora, the genus Actinocrispum, the genus Kutzneria, the genus Lentzea, the genus Kibdelosporangium, the genus Catenulispora, the genus Planomonospora, the genus Dyella, the genus Marmoricola, the genus Actinosynnema, the genus Prauserella, and the genus Yuhushiella. An oxidoreductase having high sequence identity (e.g., 50% or more, 51% or more, 52% or more, 53% or more, 54% or more, 55% or more, 56% or more, 57% or more, 58% or more, 59% or more, 60% or more, 61% or more, 62% or more, 63% or more, 64% or more, 65% or more, 66% or more, 67% or more, 68% or more, 69% or more, 70% or more, 71% or more, 72% or more, 73% or more, 74% or more, 75% or more, 76% or more, 77% or more, 78% or more, 79% or more, 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more, e.g., 99% or more) with respect to the amino acid sequence of the oxidoreductase described in SEQ ID NO: 1, and oxidoreductase having an amino acid sequence in which 1 or more amino acids are modified or mutated, deleted, substituted, added and/or inserted in the amino acid sequence of SEQ NO: 1.

In one embodiment, the dehydrogenase may be the oxidase derived from the F2 strain of the genus Arthrobacter or the oxidase produced by E. coli transformed with a plasmid containing the oxidase gene derived from the F2 strain of the genus Arthrobacter. The oxidase can be efficiently expressed in large quantities by using E. coli transformed with the plasmid containing the oxidase gene derived from the F2 strain of the genus Arthrobacter.

In one embodiment, a reaction condition of the dehydrogenase may be any condition as long as it is a condition for acting on vitamin D derivative and efficiently catalyzing a dehydrogenation reaction. Generally, an enzyme has an optimum temperature and optimum pH that exhibit the highest activity. Therefore, it is suitable that the reaction condition is near the optimum temperature and the optimum pH.

In one embodiment, various chemicals may participate in the reaction process of the dehydrogenase when the dehydrogenase of the present invention acts on the vitamin D derivative. For example, when the dehydrogenase of the present invention acts on the vitamin D derivative, the movement of electrons may participate in the redox reaction.

In one embodiment, a method for measuring vitamin D derivative is provided in which the dehydrogenase is acted on vitamin D derivative to oxidize vitamin D derivative, and the withdrawn electron reduces the mediator, and the reduced mediator further reacts with a reagent which undergoes coloring or fading. Examples of the colorimetric substrate used in the present invention include, for example, tetrazolium compounds (Tetrazolium blue, Nitro-tetrazolium blue, Water soluble tetrazolium (WST)-1, WST-3, WST-4, WST-5, WST-8, WST-9) and the like in addition to DCIP (2, 6-Dichlorophenolindophenol).

In an embodiment, when quantification of vitamin D derivative using blood as a sample, the sample may be arbitrarily selected from whole blood, plasma, or serum depending on the vitamin D derivative to be measured. In addition, dehydrogenase or a composition for quantification of vitamin D derivative containing dehydrogenase may be directly mixed with a sample, or a sample may be pretreated before mixing with dehydrogenase or the composition for quantification of vitamin D derivative containing dehydrogenase. For example, vitamin D binding protein may be degraded with protease to release vitamin D derivative and then mixed with dehydrogenase or a composition for quantification of vitamin D derivative containing dehydrogenase.

### [Method for Preparing Enzymes]

Since the dehydrogenase according to the present invention can be prepared by the same preparation method as that of the above-described oxidoreductase, a detailed description thereof will be omitted.

### [Enzyme Activity Measurement]

The method for measuring the activity of the enzyme may be any method as long as it directly or indirectly measures a product of a reaction catalyzed by the enzyme. For example, if a product by a reaction catalyzed by an enzyme and a reagent reacting with the product (hereinafter, a “product reaction reagent”) are reacted and an absorbing substance generated by the reaction is measured, the enzyme activity can be measured by performing absorbance measurement.

### [Composition Containing Dehydrogenase and Kit for Quantification of Vitamin D Derivative]

In an embodiment, vitamin D derivative may be quantified utilizing dehydrogenase according to the present invention. A quantification method of vitamin D derivative utilizing dehydrogenase according to the present invention may be carried out by providing a composition containing dehydrogenase and a product reaction reagent, or may be carried out by combining dehydrogenase and a commercially available product reaction reagent. For example, it may be provided as a composition for quantification of vitamin D derivative containing dehydrogenase, or a composition for quantification of vitamin D derivative further containing a mediator which is reduced by adding dehydrogenase and a reagent which reacts with the reduced mediator. It may also be provided as a kit for quantification of vitamin D derivative including dehydrogenase, a mediator reduced by the addition of dehydrogenase, and a reagent which reacts with the reduced mediator.

The mediator (also referred to as an artificial electron mediator, an artificial electron acceptor or an electron mediator) used in the measurement method or the kit for quantification of the present invention is not particularly limited as long as it can receive an electron from dehydrogenase. Examples of the mediators include quinones, phenazines, viologens, cytochromes, phenoxazines, phenothiazines, ferricyanides e.g., potassium ferricyanide, ferredoxins, ferrocene, osmium complexes and derivatives thereof, and the phenazine compounds include, but are not limited to, PMS, methoxy PMS, 5-Methylphenazinium ethylsulfate (PES), and methoxy PES.

### [Sensor Chip and Electrode]

In an embodiment, the dehydrogenase of the present invention may be applied, adsorbed, or immobilized on an electrode. Preferably, the dehydrogenase of the present invention is applied, adsorbed, or immobilized on a working electrode. Since the configuration of the electrode can be applied with the same configuration as that of the configuration described for the electrode using oxidoreductase, a detailed description thereof will be omitted. In addition, the dehydrogenase can be immobilized on the electrode by crosslinking, coating with a dialysis membrane, encapsulation in a polymer matrix, use of a photocrosslinkable polymer, use of a conductive polymer, use of an oxidation/reduction polymer, and the like.

The dehydrogenase of the present invention can be applied to various electrochemical measurement methods by using a potentiostat, a galvanostat, or the like. The electrochemical measurement includes various techniques such as amperometry, potentiometry, and coulometry. For example, in the case of the an amperometry method, the concentration of vitamin D derivative in the sample can be calculated by mixing a mediator in a reaction solution, transferring electrons generated when dehydrogenase reacts with vitamin D derivative to an oxidized mediator, generating a reduced mediator, and measuring a current value generated by applying −1000 mV to +500 mV (vs. Ag/AgCl). As the counter electrode, a carbon electrode or a platinum electrode is preferred. For example, a calibration curve can be generated by measuring current values for known concentrations of vitamin D derivative (0, 100, 200, 500 μM) and plotting against the concentrations of vitamin D derivative. The concentration of vitamin D derivative can be obtained from the calibration curve by measuring the current value of the unknown vitamin D derivative.

In addition, printed electrodes (sensor chips) can be used to reduce the amount of solution required for measurement. In this case, the electrodes are preferably formed on a substrate composed of an insulating substrate. A configuration of the sensor chip using dehydrogenase may be the same as the configuration of the sensor chip using an oxidoreductase, and a detailed description thereof will be omitted.

### [Vitamin D Derivative Measurement Sensor]

In one embodiment, a vitamin D derivative measurement sensor using dehydrogenase of the present invention is provided. The sensor is a vitamin D derivative measurement device using the dehydrogenase of the present invention and includes a sensor chip containing the dehydrogenase and a measurement unit. The configuration of the vitamin D derivative measurement sensor using dehydrogenase may be the same as the configuration of the vitamin D derivative measurement sensor using an oxidoreductase, and a detailed description thereof will be omitted.

As described above, the quantification method of vitamin D derivative, the dehydrogenase for quantification, the composition for quantification, and the kit for quantification according to the present invention can provide a novel quantification method for quantifying the concentration of vitamin D derivative, a novel enzyme for quantification, a novel composition for quantification, a novel kit for quantification, a novel electrode, a novel sensor chip, and a novel sensor by containing a dehydrogenase.

### [Quantification Method of Vitamin D Derivative Using Oxidase Activity]

The oxidase used in the present invention is an oxidizing enzyme which acts on vitamin D derivative as a substrate. However, by the time of filing the present application, no oxidase acting on vitamin D derivative has been identified.

In one embodiment, the oxidase may include an oxidase selected from the oxidoreductases described above or having high vitamin D derivative oxidase activity among the oxidoreductases described above. In one embodiment, the oxidase includes cholesterol oxidase derived from the F2 strain of the genus Arthrobacter. In one embodiment, the oxidase of the present invention includes oxidase derived from the F2 strain of the genus Arthrobacter, but also oxidase derived from microorganisms classified as the class Actinobacteria, and oxidase derived from microorganisms classified as the genus Arthrobacter. In one embodiment, the oxidase of the present invention includes an oxidoreductase derived from microorganisms classified as the genus Herbaspirillum or the genus Pedobacter. In one embodiment, the oxidase of the present invention includes an oxidase derived from a microorganism classified as the genus Corynebacterium, the genus Rhodococcus, the genus Brevibacterium, the genus Nocardia, the genus Vitiosangium, the genus Dietzia, the genus Tomitella, the genus Actinomadura, the genus Actinoallomurus. In one embodiment, the oxidase of the present invention includes an oxidoreductase derived from microorganisms classified as the genus Amycolatopsis, the genus Actinoplanes, the genus Krasilnikovia, the genus Couchioplanes, the genus Streptosporangium, the genus Nonomuraea, the genus Streptacidiphilus, the genus Nocardioides, the genus Alloactinosynnema, the genus Microbispora, the genus Actinocrispum, the genus Kutzneria, the genus Lentzea, the genus Kibdelosporangium, the genus Catenulispora, the genus Planomonospora, the genus Dyella, the genus Marmoricola, the genus Actinosynnema, the genus Prauserella, and the genus Yuhushiella. An oxidase having high sequence identity (e.g., 50% or more, 51% or more, 52% or more, 53% or more, 54% or more, 55% or more, 56% or more, 57% or more, 58% or more, 59% or more, 60% or more, 61% or more, 62% or more, 63% or more, 64% or more, 65% or more, 66% or more, 67% or more, 68% or more, 69% or more, 70% or more, 71% or more, 72% or more, 73% or more, 74% or more, 75% or more, 76% or more, 77% or more, 78% or more, 79% or more, 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more, e.g., 99% or more) with respect to the amino acid sequence of the oxidase described in SEQ ID NO: 1, and oxidoreductase having an amino acid sequence in which 1 or more amino acids are modified or mutated, deleted, substituted, added and/or inserted in the amino acid sequence of SEQ NO: 1.

In one embodiment, the oxidase may be an oxidase derived from the F2 strain of the genus Arthrobacter or the oxidase produced by E. coli transformed with a plasmid containing the oxidase gene derived from the F2 strain of the genus Arthrobacter. The oxidase can be efficiently expressed in large quantities by using E. coli transformed with the plasmid containing the oxidase gene derived from the F2 strain of the genus Arthrobacter.

In one embodiment, a reaction condition of the oxidase may be any condition as long as it is a condition for acting on vitamin D derivative and efficiently catalyzing an oxidation reaction. Generally, an enzyme has an optimum temperature and optimum pH that exhibit the highest activity. Therefore, it is suitable that the reaction condition is near the optimum temperature and the optimum pH.

In one embodiment, various chemicals may participate in the reaction process of the oxidase when the oxidase of the present invention acts on the vitamin D derivative. For example, when the oxidase acts on vitamin D derivative, the oxygen may participate as an electron acceptor in a redox reaction.

In one embodiment, a compound generated by reacting the oxidase with vitamin D derivative include hydrogen peroxide and the like. The amount of hydrogen peroxide generated by reacting the oxidase with vitamin D derivative can be measured by a colorimetric method utilizing, for example, a catalytic reaction of peroxidase. The compound consumed by reacting the oxidase with vitamin D derivative include oxygen and the like. The amount of oxygen consumed by reacting the oxidase with vitamin D derivative can be measured by, for example, an electrochemical method using an oxygen electrode.

In an embodiment, when quantification of vitamin D derivative using blood as a sample, the sample may be arbitrarily selected from whole blood, plasma, or serum depending on the vitamin D derivative to be measured. In addition, oxidase or a composition for quantification of vitamin D derivative containing oxidase may be directly mixed with a sample, or a sample may be pretreated before mixing with oxidase or the composition for quantification of vitamin D derivative containing an oxidase. For example, vitamin D binding protein may be degraded with protease to release vitamin D derivative and then mixed with oxidase or a composition for quantification of vitamin D derivative containing oxidase.

### [Method for Preparing Enzyme]

Since the oxidase according to the present invention can be prepared by the same preparation method as that of the above-described oxidoreductase, a detailed description thereof will be omitted.

### [Enzyme Activity Measurement]

The method for measuring the activity of the enzyme may be any method as long as it directly or indirectly measures a product of a reaction catalyzed by the enzyme. For example, if a product by a reaction catalyzed by an enzyme and a product reaction reagent are reacted and an absorbing substance generated by the reaction is measured, the enzyme activity can be measured by performing absorbance measurement.

### [Composition Containing Oxidase and Kit for Quantification of Vitamin D Derivative]

In an embodiment, vitamin D derivative may be quantified utilizing an oxidase according to the present invention. A quantification method of vitamin D derivative utilizing an oxidase according to the present invention may be carried out by providing a composition containing oxidase and a product reaction reagent, or may be carried out by combining oxidase and a commercially available product reaction reagent. For example, it may be provided as a composition for quantification of vitamin D derivative containing oxidase, or a composition for quantification of vitamin D derivative further containing a reagent which reacts with hydrogen peroxide produced by adding an oxidase. Further, it may be provided as a kit for quantification of vitamin D derivative containing oxidase and a reagent which reacts with hydrogen peroxide produced by adding oxidase.

### [Sensor Chip and Electrode]

In an embodiment, the oxidase of the present invention may be applied, adsorbed, or immobilized on an electrode. Preferably, the oxidase of the present invention is applied, adsorbed, or immobilized on a working electrode. Since the configuration of the electrode can be applied with the same configuration as that of the configuration described for the electrode using the oxidoreductase, a detailed description thereof will be omitted. In addition, the oxidase can be immobilized to the electrode by crosslinking, coating with a dialysis membrane, encapsulation in a polymer matrix, use of a photocrosslinkable polymer, use of an electrically conductive polymer, use of an oxidation/reduction polymer, and the like.

The oxidase of the present invention can be applied to various electrochemical measurement method by using a potentiostat, a galvanostat, or the like. The electrochemical measurement includes various techniques such as amperometry, potentiometry, and coulometry. For example, in the case of the amperometry method, the concentration of vitamin D derivative in a sample can be calculated by measuring a current value generated by applying +600 mV to +1000 mV (vs. Ag/AgCl) by a hydrogen peroxide electrode to hydrogen peroxide produced when oxidase reacts with vitamin D derivative. For example, a calibration curve can be generated by measuring current values for known concentrations of vitamin D derivative (0, 5, 10, 50 μM) and plotting against the concentrations of vitamin D derivative. The concentration of vitamin D derivative can be obtained from the calibration curve by measuring the current value of the unknown vitamin D derivative. As the hydrogen peroxide electrode, for example, a carbon electrode or a platinum electrode can be used. The amount of hydrogen peroxide can be quantified by measuring the reduction current value generated by applying −400 mV to +100 mV (vs. Ag/AgCl) using an electrode immobilized with a reductase such as a peroxidase or catalase, instead of the hydrogen peroxide electrode, and the value of the vitamin D derivative can also be measured.

In addition, printed electrodes (sensor chips) can be used to reduce the amount of solution required for measurement. In this case, the electrodes are preferably formed on a substrate composed of an insulating substrate. A configuration of the sensor chip using an oxidase may be the same as the configuration of the sensor chip using an oxidoreductase, and a detailed description thereof will be omitted.

### [Vitamin D Derivative Measurement Sensor]

In one embodiment, a vitamin D derivative measurement sensor using oxidase of the invention is provided. The sensor is a vitamin D derivative measurement device using the oxidase of the present invention and includes a sensor chip containing the oxidase and a measurement unit. The configuration of the vitamin D derivative measurement sensor using oxidase may be the same as the configuration of the vitamin D derivative measurement sensor using an oxidoreductase, and a detailed description thereof will be omitted.

As described above, the quantification method of vitamin D derivative, the oxidase for quantification, the composition for quantification, and the kit for quantification according to the present invention can provide a novel quantification method for quantifying the concentration of vitamin D derivative, a novel enzyme for quantification, a novel composition for quantification, a novel kit for quantification, a novel electrode, a novel sensor chip, and a novel sensor by containing an oxidase.

### Example 1

By showing specific examples and test results of the quantification method, the oxidoreductase for quantification, the composition for quantification, and the kit for quantification according to the present invention described above, a detailed description will be given.

[Preparation of Recombinant Plasmid pUC18-ChoF]

The DNA fragment of the vector pUC18 was amplified by PCR using SEQ ID NOs: 3 and 4 as the primers and pUC18 as the template. 1.0 μl of DpnI (manufactured by New England BioLabs, Inc.) was added to the PCR reaction solution and treated for 1 hour at 37° C., followed by agarose gel electrophoresis, and the gel containing the target DNA fragment (about 2.7 kbp) was cutout. The target DNA fragment was extracted from the gel using illustra (registered trademark) GFX PCR DNA and Gel Band Purification Kit (manufactured by GE Healthcare).

A ChoF gene having a base sequence of SEQ ID NO: 2 was entrusted to Integrated DNA Technologies by dividing into the first half portion (choF-f1) described in SEQ ID NO: 5 and the second half portion (choF-f2) described in SEQ ID NO: 6. 15 bases at the 3′ end of choF-f1 and 15 bases at the 5′ end of choF-f2 (ACAACCTTGCATCGC) indicate overlapping sequence in the first and second half of ChoF gene. The ChoF gene according to this example is the cholesterol oxidase (ChoF, UniprotKB Entry name Q56DL0-9MICC) derived from the F2 strain of the genus Arthrobacter deleting N-terminal 45 a.a. predicted as a signal peptide.

In-fusion reaction (50° C., 15 minutes) was performed according to In-Fusion (registered trademark) HD Cloning Kit manual using the DNA fragment of the vector pUC18 and two ChoF gene fragments to obtain the plasmid (pUC18-ChoF) for expression of ChoF. An E. coli JM109 strain was transformed with the resulting plasmid.

### [Expression of ChoF]

The E. coli JM109 (pUC18-ChoF) strain with the recombinant plasmid was inoculated into 2.5 ml of LB-amp medium [1% (W/V) bactotryptone, 0.5% (W/V) yeast extract, 0.5% (W/V) NaCl, 50 μg/ml Ampicillin], and cultured by shaking at 37° C. for 24 hours to obtain the seed culture solution.

1 ml of the seed culture solution was inoculated into 50 ml of LB-amp medium [1% (W/V) bactotryptone, 0.5% (W/V) yeast extract, 0.5% (W/V) NaCl, 50 μg/ml Ampicillin] containing 0.1 mM IPTG charged into a Sakaguchi flask and cultured at 25° C. for 16 hours.

The culture solution was centrifuged at 6,500×g for 10 minutes to collect bacterial cells. The obtained bacterial cells were washed with 10 mM potassium phosphate buffer (pH 7.0) and resuspended. After ultrasonic pulverization of the bacterial cell suspension, the supernatant obtained by centrifuging at 20,400×g for 15 minutes was dialyzed with 10 mM potassium phosphate buffer (pH 6.0) using Amicon (registered trademark) Ultra Ultracel-30K (manufactured by Millipore) to obtain the crude enzyme solution of ChoF.

### [Purification of ChoF]

The crude enzyme solution of ChoF was injected into a HiScreen Capto Q (manufactured by GE Healthcare, resin volume 4.7 ml) equilibrated with 10 mM potassium phosphate buffer pH 6.0 to recover fractions that did not bind to the anion exchange column.

After dialyzing the recovered fractions with 10 mM CHES-NaOH buffer (pH 9.5) with Amicon Ultra Ultracel-30K, the recovered fractions were injected into HiScreen Capto Q (manufactured by GE Healthcare, resin volume 4.7 ml) equilibrated with 10 mM CHES-NaOH buffer (pH9.5) to bind to the anion exchange column. Thereafter, the column was washed with 10 mM CHES-NaOH buffer (pH 9.5), and ChoF bound to the column was eluted with 10 mM CHES-NaOH buffer (pH 9.5) containing NaCl having a concentration gradient of 0 mM to 250 mM.

The eluted fraction was concentrated using Amicon Ultra Ultracel-30K. The eluted fraction was fractionated by HiLoad 26/60 Superdex 200 (manufactured by GE Healthcare) equilibrated with 10 mM potassium phosphate buffer (pH 7.0) containing 150 mM NaCl. The purity of each eluted fraction was assessed by polyacrylamide gel electrophoresis (SDS-PAGE), and the fraction containing no contaminant protein was collected and used as a purified preparation of ChoF.

A protein concentration of the purified ChoF was determined by the ultraviolet absorption method utilizing absorbance at 280 nm (A280) (see Protein Sci. 4, 2411-23, 1995). The molecular weight of ChoF calculated from the amino acid sequence is 55.6 kDa. Since ChoF contains 19 residues of tyrosine and 9 residues of tryptophan, A280 of 1.0 mg/ml of ChoF solutions indicates 1.4.

### [Quantification of Vitamin D Derivative by Oxidase Activity]

Using the ChoF obtained by the above-described method, oxidase activity was measured using 25-hydroxyvitamin D₃ (calcidiol), which is vitamin D derivative, as a substrate.

### [Substrate Reagent]

4.19 mg of 25-hydroxyvitamin D₃ was dissolved in 1 ml DMSO (final concentration 10 mM). Since 25-hydroxyvitamin D₃ is soluble in fats, it was diluted to 5, 10, 20, and 100 μM using 3.9% (v/v) TritonX-100 solutions to maintain solubility.

TABLE 1

97.2 mM Potassium phosphate buffer (pH 7.0)
13 mg/ml ChoF
1.0 mM 4-Aminoantipyrine (4-AA, manufactured by Fujifilm Wako
Pure Chemical Corporation)
1 mM N-ethyl-N-(2-hydroxy-3-sulfopropyl)-3-methylaniline
(TOOS, manufactured by DOJINDO LABORATORIES)
3.3 U/ml Peroxidase (POD, manufactured by TOYOBO Co., LTD.)

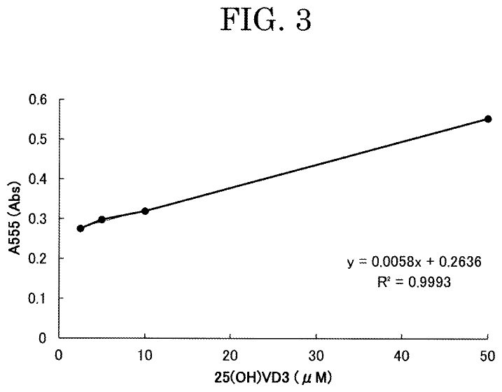

After incubation of 450 μl of the measurement reagent consisting of the composition of Table 1 for 5 minutes at 37° C., 450 μl of substrate reagent was added (measured concentrations of substrate of 2.5, 5, 10, and 50 μM), and the absorbance (Abs) at a wavelength of 555 nm was measured using a spectrophotometer (U-3900, manufactured by Hitachi High-Tech Science Corporation). The relationship between the concentration of vitamin D derivative and absorbance (A₅₅₅) after reacting for 5 minutes is shown in FIG. 3.

A result that there was a correlation between the concentration of vitamin D derivative and absorbance was obtained because the coefficient of determination (R²), which is an indicator of the correlation between the concentration of vitamin D derivative and absorbance, was 0.9993 in the range of 2.5 μM to 50 μM. Therefore, it has been shown that the oxidase activity of ChoF could be used to quantify vitamin D derivative.

### Example 2

[Preparation of Recombinant Plasmid pKK223-3 HeGMCOR]

The DNA fragment of the vector pKK223-3 was amplified by PCR using primer show in SEQ ID NO: 7 and SEQ ID NO: 8 and pKK223-3 as the template. 1.0 μl of DpnI (manufactured by New England BioLabs, Inc.) was added to a PCR reaction solution and treated for 1 hour at 37° C., followed by agarose gel electrophoresis, and the gel containing the target DNA fragment (about 4.6 kbp) was cutout. The target DNA fragment was extracted from the gel using illustra (registered trademark) GFX PCR DNA and Gel Band Purification Kit (manufactured by GE Healthcare).

A HeGMCOR gene having a base sequence of SEQ ID NO: 9 was entrusted to Integrated DNA Technologies by dividing into a first half portion (HeGMCOR-f1) described in SEQ ID NO: 10 and a second half portion (HeGMCOR-f2) described in SEQ ID NO: 11. 15 bases at the 3′ end of HeGMCOR-f1 and 15 bases at the 5′ end of HeGMCOR-f2 (GAAGGAAATGGCTAT) indicate overlapping sequence in the first and second half of HeGMCOR gene. The HeGMCOR gene according to this example is a glucose-methanol-choline family oxidoreductase (HeGMCOR, NCBI Reference Sequence: WP_094565544.1) derived from the meg3 strain of the genus Herbaspirillum, and the identity of the amino acid sequence with ChoF of Example 1 is 50%.

In-fusion reaction (50° C., 15 minutes) was performed according to In-Fusion (registered trademark) HD Cloning Kit manual using the DNA fragment of the vector pKK223-3 and two HeGMCOR gene fragments to obtain the plasmid (pKK223-3_HeGMCOR) for expression of HeGMCOR. An E. coli BL21 (DE3) was transformed with the resulting plasmid.

### [Expression of HeGMCOR]

The E. coli BL21 (DE3) (pKK223-3_HeGMCOR) strain with the recombinant plasmid was inoculated into 2.5 ml of LB-amp medium [1% (W/V) bactotryptone, 0.5% (W/V) yeast extract, 0.5% (W/V) NaCl, 50 μg/ml Ampicillin], and cultured by shaking at 37° C. for 24 hours to obtain the seed culture solution.

1.5 ml of seed culture solution was inoculated into 150 ml of LB-amp medium [1% (W/V) bactotryptone, 0.5% (W/V) yeast extract, 0.5% (W/V) NaCl, 50 μg/ml Ampicillin] containing 0.1 mM IPTG charged into a Sakaguchi flask and cultured at 25° C. for 20 hours.

The culture solution was centrifuged at 6,500×g for 10 minutes to collect bacterial cells. The obtained bacterial cells were washed with 12 ml of 10 mM potassium phosphate buffer (pH 7.0) and resuspended. After ultrasonic pulverization of the bacterial cell suspension, the supernatant obtained by centrifuging at 20,400×g for 15 minutes was collected as a cell extract. Ammonium sulfate was added to the cell extract so as to have a 35% saturation concentration, the mixture was sufficiently vortexed to dissolve the ammonium sulfate, and the supernatant obtained by centrifuged at 20,360×g for 5 minutes was used as a crude enzyme solution.

### [Purification of HeGMCOR]

Subsequently, the crude enzyme solution was injected into a HiScreen Butyl HP (manufactured by GE Healthcare, resin volume 4.7 ml) equilibrated with 10 mM potassium phosphate buffer (pH 7.5) containing 500 mM ammonium sulfate to bind to the column. Thereafter, the column was washed with 10 mM potassium phosphate buffer (pH 7.5) containing 250 mM ammonium sulfate, and HeGMCOR bound to the column was eluted with 10 mM potassium phosphate buffer (pH 7.5) containing ammonium sulfate having a concentration gradient of 250 mM to 0 mM. The purity of each eluted fraction was assessed by polyacrylamide gel electrophoresis (SDS-PAGE) to recover fractions with less contaminant proteins.

The resulting eluted fractions were concentrated using Amicon Ultra Ultracel-30K. The eluted fraction was fractionated by HiLoad 26/60 Superdex 200 (manufactured by GE Healthcare) equilibrated with 10 mM potassium phosphate buffer (pH 7.0) containing 150 mM NaCl. The purity of each eluted fraction was assessed by polyacrylamide gel electrophoresis (SDS-PAGE), and the fraction containing no contaminant protein was collected and used as a purified preparation of HeGMCOR.

A protein concentration of the purified HeGMCOR was determined by the ultraviolet absorption method utilizing absorbance at 280 nm (A280) (see Protein Sci 4, 2411-23, 1995). The molecular weight of HeGMCOR calculated from the amino acid sequence is 54.3 kDa. Since HeGMCOR contains 22 residues of tyrosine and 6 residues of tryptophan, A280 of 1.0 mg/ml of HeGMCOR solutions indicates 1.2.

### [Quantification of Vitamin D Derivative by Oxidase Activity]

Using the HeGMCOR obtained by the above-described method, oxidase activity was measured using 25-hydroxyvitamin D₃ (calcidiol), which is vitamin D derivative, as a substrate.

### [Substrate Reagent]

4.19 mg of 25-hydroxyvitamin D₃ was dissolved in 1 ml DMSO (final concentration 10 mM). Since 25-hydroxyvitamin D₃ is soluble in fats, it was diluted to 2, 5, and 20 μM using 3.9% (v/v) TritonX-100 solutions to maintain solubility.

TABLE 2

86.2 mM Potassium phosphate buffer (pH 7.0)
0.41 mg/ml HeGMCOR
1.0 mM 4-Aminoantipyrine (4-AA, manufactured by Fujifilm Wako
Pure Chemical Corporation)
1 mM N-ethyl-N-(2-hydroxy-3-sulfopropyl)-3-methylaniline
(TOOS, manufactured by DOJINDO LABORATORIES)
3.36 U/ml Peroxidase (POD, manufactured by TOYOBO Co., LTD.)

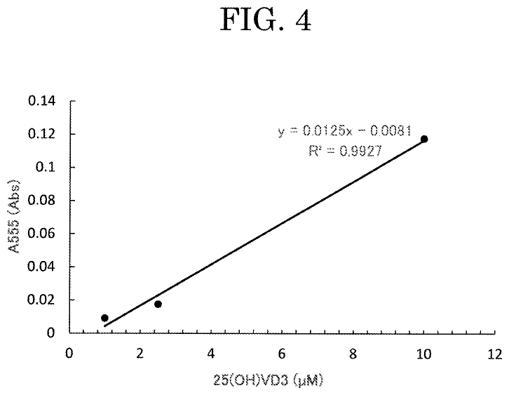

After incubation of 375 μl of the measurement reagent consisting of the composition of Table 2 for 5 minutes at 37° C., 375 μl of substrate reagent was added (measured concentrations of 1, 2.5, and 10 μM), and the absorbance (Abs) at a wavelength of 555 nm was measured using a spectrophotometer (U-3900, manufactured by Hitachi High-Tech Science Corporation). The relationship between the concentration of vitamin D derivative and absorbance (A₅₅₅) after reacting for 5 minutes is shown in FIG. 4.

A result that there was a correlation between the concentration of vitamin D derivative and absorbance was obtained because the coefficient of determination (R²), which is an indicator of the correlation between the concentration of vitamin D derivative and absorbance, was 0.9927 in the range of 1 μM to 10 μM. Therefore, it has been shown that the oxidase activity of HeGMCOR could be used to quantify vitamin D derivative.

### Example 3

### [Quantification of Vitamin D Derivative by Dehydrogenase Activity]

Using the ChoF obtained in Example 1, dehydrogenase activity was measured using 25-hydroxyvitamin D₃ (calcidiol), which is vitamin D derivative, as a substrate.

### [Substrate Reagent]

4.19 mg of 25-hydroxyvitamin D₃ was dissolved in 1 ml DMSO (final concentration 10 mM). Since 25-hydroxyvitamin D₃ is soluble in fats, it was diluted to 200, 400, 500, and 1000 μM using 3.9% (v/v) TritonX-100 solutions to maintain solubility.

TABLE 3

98.7 mM Potassium phosphate buffer (pH 7.0)
0.78 mg/ml ChoF
1.0 mM 2,6-Dichloroindophenol sodium salt hydrate (DCIP,
manufactured by Sigma-Aldrich)
0.35 mM 1-Methoxy-5-methylphenazinium methylsulfate (1-
mPMS, manufactured by DOJINDO LABORATORIES)

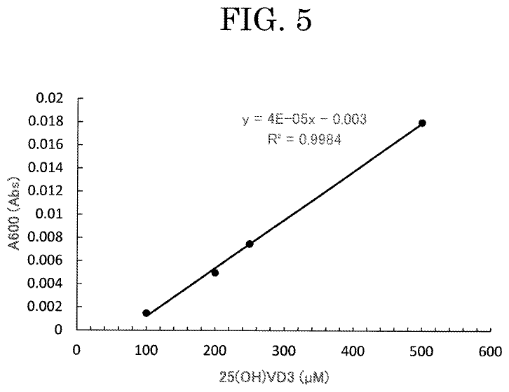

After incubation of 375 μl of the measurement reagent consisting of the composition of Table 3 for 5 minutes at 37° C., 375 μl of substrate reagent was added (measured concentrations of 100, 200, 250, and 500 μM), and the absorbance (Abs) at a wavelength of 600 nm was measured using a spectrophotometer (U-3900, manufactured by Hitachi High-Tech Science Corporation). The relationship between the concentration of vitamin D derivative after 3 minutes of reaction and the amount of change in absorbance (A₆₀₀) per minute is shown in FIG. 5.

A result that there was a correlation between the concentration of vitamin D derivative and absorbance was obtained because the coefficient of determination (R²), which is an indicator of the correlation between the concentration of vitamin D derivative and absorbance, was 0.9984 in the range of 100 μM to 500 μM. Therefore, it has been shown that the ChoF dehydrogenase activity could be used to quantify vitamin D derivative.

As described above, a novel quantification method for quantifying the concentration of vitamin D derivative, an novel enzyme for quantification, a novel composition for quantification, a novel kit for quantification, an novel electrode, a novel sensor chip, and a novel sensor can be provided by a quantification method for quantifying the concentration of vitamin D derivative by adding an oxidase to a sample containing vitamin D derivative, an oxidase for quantification of vitamin D derivative added to a sample containing vitamin D derivative, a composition for quantification of vitamin D derivative containing an oxidase added to a sample containing vitamin D derivative, a kit for quantification of vitamin D derivative containing an oxidase added to a sample containing vitamin D derivative according to the present invention. In addition, a novel quantification method for quantifying the concentration of vitamin D derivative, an novel enzyme for quantification, a novel composition for quantification, a novel kit for quantification, an novel electrode, a novel sensor chip, and a novel sensor can be provided by a quantification method for quantifying the concentration of vitamin D derivative by adding an oxidoreductase to a sample containing vitamin D derivative, an oxidoreductase for quantification of vitamin D derivative added to a sample containing vitamin D derivative, a composition for quantification of vitamin D derivative containing an oxidoreductase added to a sample containing vitamin D derivative, a kit for quantification of vitamin D derivative containing an oxidoreductase added to a sample containing vitamin D derivative according to the present invention.

### Advantageous Effects of Invention

According to the present invention, there are provided a novel quantification method for measuring 25-hydroxyvitamin D, which is vitamin D derivative, an enzyme for quantification, a composition for quantification, a kit for quantification, an electrode, a sensor chip, and a sensor.

What is claimed is:

1. A quantitation method of vitamin D derivative comprising:
adding an oxidoreductase directly oxidizing or reducing vitamin D derivative to a sample.

2. The quantitation method of vitamin D derivative according to claim 1 further comprising:
reducing a mediator by adding the oxidoreductase; and
reacting the reduced mediator with a reagent to determine a concentration of the vitamin D derivative.

3. The quantitation method of vitamin D derivative according to claim 1, wherein
the oxidoreductase is an oxidase; and
hydrogen peroxide produced or oxygen consumed by adding the oxidase is quantified to determine a concentration of the vitamin D derivative.

4. The quantitation method of vitamin D derivative according to claim 3, wherein
the oxidoreductase is an oxidase; and
hydrogen peroxide produced by adding the oxidase is reacted with a reagent to determine a concentration of the vitamin D derivative.

5. An oxidoreductase, wherein the oxidoreductase is used in the quantitation method of vitamin D derivative according to claim 1.

6. An oxidoreductase, wherein the oxidoreductase is used in the quantitation method of vitamin D derivative according to claim 2.

7. An oxidoreductase, wherein the oxidoreductase is used in the quantitation method of vitamin D derivative according to claim 3.

8. An oxidoreductase, wherein the oxidoreductase is used in the quantitation method of vitamin D derivative according to claim 4.

9. The oxidoreductase according to claim 5, wherein the oxidoreductase is an oxidoreductase belonged to EC No. 1.1.

10. The oxidoreductase according to claim 5, wherein the oxidoreductase is an oxidoreductase belonged to EC No. 1.1.3.

11. A composition for quantification of vitamin D derivative comprising: the oxidoreductase according to claim 5.

12. A composition for quantification of vitamin D derivative comprising: the oxidoreductase according to claim 9.

13. A composition for quantification of vitamin D derivative comprising: the oxidoreductase according to claim 10.

14. The composition for quantification of vitamin D derivative according to claim 11, further comprising:
a mediator to be reduced by adding the oxidoreductase; and
a reagent to be reacted with the reduced mediator.

15. The composition for quantification of vitamin D derivative according to claim 11, wherein
the oxidoreductase is an oxidase; and
the composition additionally includes a reagent reacting with hydrogen peroxide produced by adding the oxidase.

16. A kit for quantification of vitamin D derivative comprising:
the oxidoreductase according to claim 5,
a mediator reduced by adding the oxidoreductase, and
a reagent reacting with the reduced mediator.

17. A kit for quantification of vitamin D derivative comprising:
the oxidoreductase according to claim 5, and
a reagent reacting with hydrogen peroxide.

18. An electrode comprising the oxidoreductase according to claim 5.

19. A sensor chip comprising the electrode according to claim 18 as a working electrode.

20. A sensor comprising the sensor chip according to claim 19.
